(19) United States
(12) Patent Application Publication
(10) Pub. No.: US 2005/0095229 A1
(43) Pub. Date: May 5, 2005

(54) Artificial organ

(76) Inventors: Patrick Connelly, Rochester, NY (US); Michael L. Weiner, Webster, NY (US)

Correspondence Address: HOWARD J. GREENWALD P.C., 349 W. COMMERCIAL STREET SUITE 2490, EAST ROCHESTER, NY, 14445-2408, US

(21) Appl. No.: 11/003,562

(22) Filed: Dec. 2, 2004

Related U.S. Application Data
(63) Continuation of application No. 10/208,288, filed on Jul. 30, 2002.
Application No. 10/208,288 is a continuation-in-part of application No. 10/131,361, filed on Apr. 24, 2002.
Application No. 10/208,288 is a continuation-in-part of application No. 09/918,078, filed on Jul. 30, 2001, now Pat. No. 6,743,190.
Application No. 10/208,288 is a continuation-in-part of application No. 09/918,076, filed on Jul. 30, 2001, now Pat. No. 6,793,642.
Application No. 10/208,288 is a continuation-in-part of application No. 09/800,823, filed on Mar. 7, 2001, now Pat. No. 6,750,055.
Application No. 10/208,288 is a continuation-in-part of application No. 09/850,250, filed on May 7, 2001, now Pat. No. 6,488,704.
Application No. 10/208,288 is a continuation-in-part of application No. 09/852,876, filed on May 10, 2001, now abandoned.
(60) Provisional application No. 60/308,628, filed on Jul. 30, 2001.

(51) Int. Cl. C12N 5/08; A61K 45/00

(52) U.S. Cl. 424/93.7; 435/69.1; 435/366

(57) ABSTRACT

Disclosed is an artificial organ comprised of a conduit adapted to be connected to a blood pool, the blood pool comprising a first analyte and a second analyte; a cell culture media comprised of a first cell culture and a second cell culture, each of which are capable of producing the first analyte and said second analyte respectively; and a controller that is capable of measuring a concentration of the first and second analyte in the blood pool, and delivering a specified amount of the first and second analyte from the cell culture media to the blood pool, and delivering a specified amount of the second analyte from the cell culture media to the blood pool.

### CROSS-REFERENCE TO RELATED PATENT APPLICATIONS

This application is a continuation of co-pending patent application U.S. Ser. No. 10/208,288, filed on Jul. 30, 2002, which is a continuation-in-part of co-pending application U.S. Ser. No. 10/131,361 filed on Apr. 24, 2002, U.S. Ser. No. 09/918,078 filed on Jul. 30, 2001, which issued on Jun. 1, 2004 as U.S. Pat. No. 6,743,190, co-pending application U.S. Ser. No. 09/918,076 filed on Jul. 30, 2001, and claims priority from provisional application U.S. Ser. No. 60/308,628 filed on Jul. 30, 2001, U.S. Ser. No. 09/850,250 filed on May 7, 2001, which issued on Dec. 3, 2002 as U.S. Pat. No. 6,488,704, U.S. Ser. No. 09/852,876 filed on May 10, 2001, now abandoned, co-pending application U.S. Ser. No. 09/800,823 filed on Mar. 7, 2001, which issued on Jun. 15, 2004 as U.S. Pat. No. 6,750,055. The entire content of each one of the above referenced patents and patent applications is hereby incorporated by reference into this specification.

### FIELD OF THE INVENTION

This invention pertains to, in one embodiment, an artificial organ for delivering a chemical within a living body. The organ is comprised of a cell culture, a means for controlling the cell culture, and a means for delivering one or more chemicals produced by the cell culture.

### BACKGROUND OF THE INVENTION

U.S. Pat. No. 6,001,647 to Ammon B. Peck et al. (In vitro growth of functional islets of Langerhans and in vivo uses thereof), discloses a process for producing insulin in vitro. According to the patentees, the process of this patent may be used to produce insulin within the body of a diabetic patient.

Diabetes is a major public health problem. Diabetics lack the ability of normal human beings of regulating the glucose concentrations within their blood by producing and ceasing the production of insulin, as appropriate.

Insulin is necessary for the sustaining of life. Without its production, blindness may be produced, hyperketosis may be produced, brain cells may be killed, and the vascular system may be damaged. However, the presence of insulin in the blood is a mixed blessing. Too much insulin, a condition known as hyperinsulimia, has been known to cause premature aging, arthritis, and cancer.

The process of the Peck et al. patent produces insulin regardless of whether it is needed by a particular patient. In such a process, islet producing stem cells continually produce insulin. When an apparatus embodying the Peck et al. process is incorporated into a living body, the insulin so produced continually permeates into the blood supply; in Example 12 of such patent, the use of a “permeable encapsulant” is disclosed.

As will be apparent, when insulin is continually discharged into a human body, a point will come when it is no longer serving the function of regulating glucose levels in the body, and, after this point, the adverse effects of hyperinsulimia will occur. It is not natural, or desirable, for a person to continually have high levels of insulin in his or her blood.

It is an object of this invention to provide an apparatus capable of homeostatically regulating the level of insulin in a living organism.

It is another object of this invention to provide an apparatus for homeostatically regulating various other hormones within a living organism.

### SUMMARY OF THE INVENTION

In accordance with this invention, there is provided an apparatus for regulating the concentration of insulin within the blood of a living organism. This apparatus is comprised of a cell culture for producing insulin, means for measuring the concentration of glucose within the blood of the living organism, means for measuring the concentration of insulin within the blood of the living organism, means for delivering a specified amount of insulin the blood of the living organism, and means for reducing the amount of insulin within the blood of the living organism.

### BRIEF DESCRIPTION OF THE DRAWINGS

The invention will be described by reference to this specification and the drawings, in which like numerals refer to like elements, and in which:

FIG. 1 is a flow diagram of one embodiment of the process of this invention,

FIG. 2 is a schematic representation of one assembly of this invention, and

FIG. 3 is a schematic representation of another assembly of this invention.

### DESCRIPTION OF THE PREFERRED EMBODIMENTS

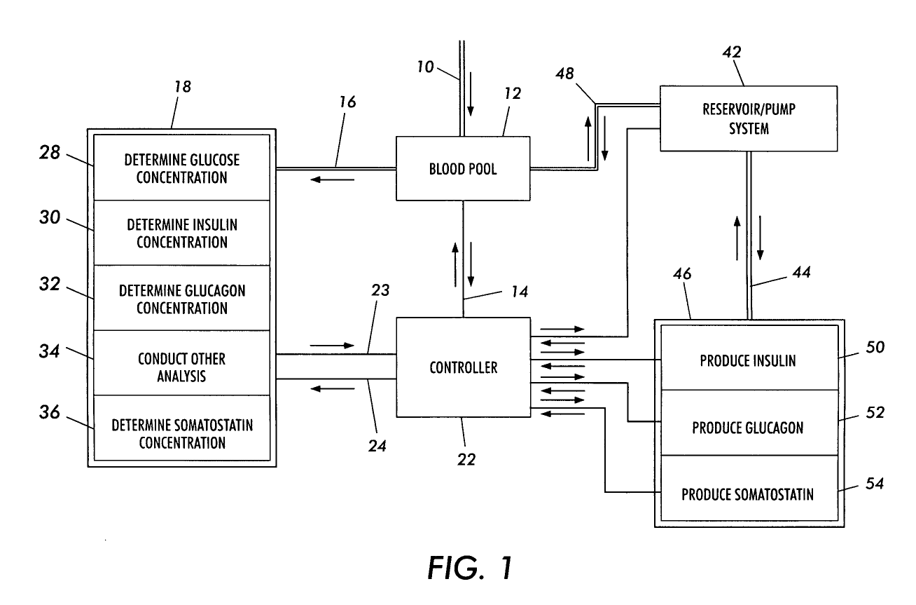

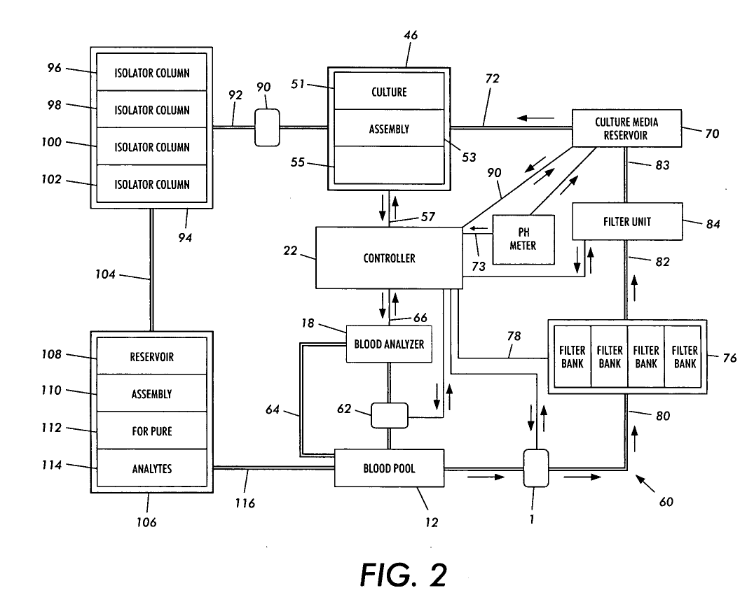

FIG. 1 is a flow diagram of one embodiment of the invention. In the first step of the process depicted, the blood of a living organism is fed via line 10 to blood pool 12. Blood may be supplied to blood pool 12 by any one of several means. Thus, e.g., one may withdraw blood from a human body by means of a hypodermic needle; in this case, the process of the invention may be practiced outside the living organism, except to the extent that blood is returned to the organism via line 14. Thus, e.g., one may implant a device, such as the device depicted in FIG. 2, within the living organism and collect blood from such organism within an in vivo reservoir; in this case, the process of the invention may be practiced entirely in the body. Thus, e.g., one may sample blood by one or more of the procedures and devices described in U.S. Pat. No. 6,159,164 to Neese (Blood Sampling System); U.S. Pat. No. 5,902,253 to Pfeiffer (Apparatus for Analyzing Body Fluids); U.S. Pat. No. 5,759,160 to Neese (Blood Sampling System); U.S. Pat. No. 5,387,192 to Glantz (Hybrid Portal); U.S. Pat. No. 4,871,351 to Feingold (Implantable Medication Infusing System); U.S. Pat. No. 4,832,034 to Pizziconi (Method and Apparatus for Withdrawing, Collecting, and Biosensing Chemical Constituents from Complex Fluids); and the like; the entire disclosure of each of these U.S. patents is hereby incorporated by reference into this specification.

Referring again to FIG. 1, a portion of the blood in the blood pool 12 is fed via line 16 to anaylzer 18. In analyzer 18, one or more blood parameters may be analyzed in response to a signal from controller 22 fed via line 24. The information obtained by such analyses is returned to the controller 22 which, in response to such information, may activate an artificial organ function and/or may take or cause to be taken one or more other actions.

In one embodiment, illustrated in FIG. 1, the controller 22 causes the analyzer 18 to determine the concentration of glucose within the blood sample; this is done in step 28. The analysis of the glucose concentration in the blood may be conducted by conventional means such as, e.g., by a glucose sensor assembly. By way of illustration and not limitation, one may use the processes and devices described in U.S. Pat. No. 5,660,163 to Schulman (Glucose Sensor Assembly); U.S. Pat. No. 5,448,992 to Kupershmidt (Method and Apparatus for Non-invasive Phase Sensitive Measurement of Blood Glucose Concentration); U.S. Pat. No. 5,995,860 to Sun (Implantable Sensor and System for Measurement and Control of Blood Constituent Levels); U.S. Pat. No. 6,175,752 to Say (Analyte Monitoring Device and Methods of Use); U.S. Pat. No. 6,162,611 to Heller (Subcutaneous Glucose Electrode); U.S. Pat. No. 6,143,164 to Heller (Small Volume in Vitro Analyte Sensor), and the like. The disclosure of each of these U.S. patents is hereby incorporated by reference into this specification.

In step 30 of the process depicted in FIG. 1, the insulin concentration of the blood sample is determined. In step 32 of the process, the glucagon concentration of the blood sample is determined. The determinations may be made in accordance with prior art procedures and devices. Thus, e.g., one may use one or more of the procedures and devices described, e.g., in U.S. Pat. No. 4,792,597 to Gaudiana (Melt-processable Polyesteramides having para-linked, substituted-phenylene radicals); U.S. Pat. No. 5,070,025 to Klein (Process for the Determination of a Protein According to Fluorescence Polarization Immunoassay Principle); U.S. Pat. No. 6,180,336 to Osbourn (Labelling and Selection of Molecules); U.S. Pat. No. 6,002,000 to Singh (Chemiluminscent Compounds and Method of Use); U.S. Pat. No. 5,936,070 to Singh (Chemiluminescent Compounds and Methods of Use); and the like. The disclosure of each of these U.S. patents is hereby incorporated by reference into this specification.

Referring again to FIG. 1, other analysis or analyses may optionally be conducted in step 34 of the process. Thus, by way of illustration and not limitation, one can analyze the expression of certain blood factors which are known or believed to cause disease. In step 36 of the process, which is optional, the concentration of somatostatin is determined.

As is known to those skilled in the art, somatostain inhibits the secretion of both insulin and glucagon, as well as growth hormone and thyroid-stimulating hormone. See, e.g., page 765 of John B. West's “Best and Taylor's Physiological Basis of Medical Practice,” Twelfth Edition (Williams and Wilkins, Baltimore, Md., 1991). Reference may also be had to U.S. Pat. No. 6,011,008 to Domb (Conjugates of Biologically Active Substances); U.S. Pat. No. 5,531,925 to Landh (Particles, Methods of Preparing said Particles and Uses Thereof); U.S. Pat. No. 5,491,131 Puyol (Somatostatin-active polypeptide Composition); U.S. Pat. No. 5,260,275 to Cooper (Hypoglycemics); and the like. The disclosure of each of these U.S. patents is hereby incorporated by reference into this specification.

As will be apparent to those skilled in the art, for proper homeostatic regulation of glucose and insulin within a living organism, glucose, insulin, glucagon, and somatstatin all must be present in specified concentrations and ratios. The process of this invention allows one to produce the conditions necessary for ideal homeostatic regulation of such analytes.

The information produced in analyzer 18 is fed to controller 22 via line 23, which produces a computer-readable profile representing the identity and relative abundance of the glucose, insulin, glucagon, and somatostatin in the blood. The controller is equipped with an algorithm which it can determine the ideal concentration of each such analyte and can thereafter cause additional insulin and/or glucagon and/or somatostatin and/or other analyte to be added to the blood pool 12.

Controllers for analyzing and regulating the composition of a biological fluid are known. Thus, e.g., in U.S. Pat. Nos. 6,064,754 to Parekh (Computer-assisted Methods and Apparatus for Identification and Characterization of Biomolecules in a Biological Sample), computer-assisted methods and devices for identifying, selecting, and characterizing biomolecules in a biological sample are disclosed. Thus, for example, one may use one or more of the processes or devices described in U.S. Pat. No. 6,185,455 to Loeb (Method of Reducing the Incidence of Medical Complications using Implantable Microstimulators); U.S. Pat. No. 6,122,536 to Sun (Implantable Sensor for Measurement and Control of Blood Constituent Levels); U.S. Pat. No. 5,995,960 to Lochner (Method and System for Improving Efficiency of Programs Utilizing Databases by Executing Scenarios Based on Recalled Processed Information); U.S. Pat. No. 5,978,713 to Prutchi (Implantable Device with Digital Waveform Telemetry); U.S. Pat. No. 5,971,931 to Raff (Biologic Micromonitoring Methods and Systems); U.S. Pat. No. 5,967,986 to Cimochowski (Endoluminal Implant with Fluid Flow Sensing Capability); and the like. The disclosure of each of these U.S. patents is hereby incorporated by reference into this specification.

In one embodiment, the controller contains a processing system utilizing an application specific integrated circuit (“ASIC”). These ASIC controllers are well known and are described, e.g., in U.S. Pat. Nos. 5,937,202 to Crosetto (High-Speed, Parallel, Processor Architecture for Front-End Electronics, Based on a Single Type of ASIC, and Method Use Thereof); U.S. Pat. No. 6,041,257 to MacDuff (Method of Using a Measuring Instrument and Data Gathering System); U.S. Pat. No. 6,165,155 to Jacobsen (Multipathway Electronically-Controlled Drug Delivery System); and the like. The entire disclosure of each of these U.S. patents is hereby incorporated by reference into this specification.

In one embodiment, the controller comprises a processor complex for processing data from at least one input, comprising at least a first and second processor, each having a data input and a data output, a data input of the second processor receiving data from the data output of the first processor; each processor being programmed with a respective algorithm for processing data received from a respective data input; said first processor being configured to receive raw data and process the raw data according to the respective algorithm programmed therein, and configured to receive other raw data and pass said other raw data to said second processor; and said second processor being configured to receive said other raw data passed from said first processor and process the other raw data according to the algorithm programmed in said second processor, and said second processor is configured to receive processed data from said first processor and pass the processed data from the data input to the data output of said second processor.

Based upon the analyses of the analytes found in the blood sample, the controller 22 will cause either insulin and/or glucagon and/or somatostatin to be withdrawn from blood pool 12 via reservoir/pump system 42 and fed via line 44 to cell culture assembly 46 cell culture assembly 46. Alternatively, or additionally, reservoir/pump system 42 can pump insulin-containing material and/or glucagon-containing material and/or somatostatin-containing material via line 48 and send it to blood pool 12. The reservoir/pump system is equipped with various filtration and separation devices so that it is capable of separating the insulin and/or glucagon and/or somatostatin from blood with which it may be admixed and returning the blood so separated to blood pool 12.

In another embodiment, the reservoir/pump system 42 is comprised of an insulin pump. Such insulin pumps are well known to those skilled in the art and are described, e.g., in U.S. Pat. No. 6,181,957 to Lambert (Non-Invasive Glucose Monitor); U.S. Pat. No. 6,168,575 to Soltanpour (Method and Apparatus for Controlling Instraocular Pressure); U.S. Pat. No. 6,165,155 to Jacobsen (Multipathway Electronically-Controlled Drug Delivery System); U.S. Pat. No. 6,162,611 to Heller (Subcutaneous Glucose Electrode); U.S. Pat. No. 6,135,978 to Houben (System for Pancreatic Stimulation and Glucose Measurement); U.S. Pat. No. 6,124,134 to Stark (Glucose Related Measurement Method and Apparatus); U.S. Pat. No. 6,123,668 to Abreu (Method and Apparatus for Signal Transmission and Detection Using a Contact Device); and the like. The entire disclosure of each of these United States patents is hereby incorporated by reference into this specification.

In yet another embodiment, the reservoir/pump system is comprised of a pump for pumping or withdrawing analytes such as insulin, glucagon, and somatostatin. One may use for this purpose conventional implantable drug delivery devices. Thus, by way of illustration and not limitation, one may use the devices disclosed in U.S. Pat. No. 5,836,985 to Rostami (Method for Treating Abnormal Atrial or Ventricular Activity); U.S. Pat. No. 5,607,418 to Arzbaecher (Implantable Drug Delivery Apparatus), and the like. Regardless of the device used, the analyte is added to or withdrawn from the blood pool as dictated by the analyses performed by the controller 22.

Reservoirs 46 include a reservoir 50 in which a tissue culture produces and accumulates insulin. Tissue cultures which produce insulin are well known.

As is known to those skilled in the art, one can grow embryonic islet of Langerhan cells with Acinar cells of the pancreas in vitro. These form a pseudo organ that can produce insulin. Murine embryonic pancreata can be dissected under a microscope. Different conditions can be applied to culture these samples to form cells, which will differentiate into functional in vitro pancreata.

Reference may be had to U.S. Pat. No. 6,110,743 to Levine (Development and use of Human Pancreatic Cell Lines) for a discussion on the creation of genetically engineered cells and their use in transplant therapy. The entire disclosure of this U.S. patent is hereby incorporated by reference into this specification.

Reference may also be had to U.S. Pat. No. RE036,844 to Jones (Cellular Attachment to Trans-Epithelial Appliances). This patent discloses a method of forming three-dimensional epithelial cellular structures with components normally derived in developing organs, and the use of 804G cells (rat bladder carcinoma cells) for the production of hemi-desmosome components that are responsible for attachment of epithelial cells to the basement membrane. In one embodiment, the implantable device is a biocompatible object (i.e. stainless steel mesh) which can be molded to any shape. The material is coated with the soluble factor from 804G cells responsible for producing ectopic hemi-desmosome formation through the attachment of any number of cells. Epithelial cell interaction with the basement membrane is a strict requirement for proper functionality of a variety of epithelial and mesenchymal cell types.

Referring again to FIG. 1, and in the preferred embodiment depicted therein, glucagon is produced by a cell culture in reservoir 52. One may produce glucagon in a cell culture, and/or another hormone in a cell culture 54 (somatostatin) by a process which comprises culturing pancreatic cells from a mammalian species in a basal nutrient medium supplemented with normal serum at below about 0.5% and glucose at below about 1 millimolar, allowing said insulin producing stem cells to grow for at least about 3 weeks, and initiating cellular differentiation into mature islet cells by re-feeding the insulin producing stem cells in culture with a nutrient medium supplemented with normal serum at about 0.5 to about 10% and glucose at about 2.5 to about 10 millimolar; see, e.g., U.S. Pat. No. 6,001,647 to Peck (In Vitro Growth of Functional Islets of Langerhans and in Vivo uses Thereof), the entire disclosure of which is hereby incorporated by reference into this specification.

A Preferred Artificial Organ

FIG. 2 is a schematic diagram of one preferred artificial organ 60 which, in one embodiment, is implantable within a living organism. Referring to FIG. 2, a source of venous blood 12 is supplied to the organ 60. The blood may be supplied from a source external to the body, such as via a blood transfusion. In one preferred embodiment, the blood is supplied by a living human body.

Means for withdrawing or segregating or channeling blood from a living organism are well known and are described in, e.g., U.S. Pat. No. 5,902,336 to Mishkin (Implantable Device and Method for Removing Fluids from the Blood of a Patient Method for Implanting such a Device and Method for Treating a Patient Experiencing Renal Failure). This patent presents a novel approach for the surgical implantation of a filtering device using filters of specified pore size and with the passage of specified flow rates.

By way of further illustration, U.S. Pat. No. 6,123,861 to Santini (Fabrication of Microchip Drug Delivery Devices) discloses the fabrication of miniaturized drug delivery systems using similar fabrication processes as those used in integrated circuit (IC) production. The devices disclosed in this patent may be used in conjunction with a source of venous blood to supply analytes (such as drugs, hormones, blood constituents, mixtures thereof, etc.) to a system.

A major hurdle in the development of artificial organ systems or in transplant therapy regimes is in the host immune response. Attempts have been made to implant in vitro organ cultures in various anatomical regions of the body in an attempt to replace loss of physiologic function.

By way of further illustration, U.S. Pat. No. 6,001,647 to Peck (Invitro Growth of Functional Islets of Langerhans and in Vivo uses Thereof) discloses in vitro culture systems, which are manipulated (with, e.g., recombinant genetic techniques) to produce functional islets of Langerhans. The implantable in vitro systems discussed in this U.S. Pat. No. 6,001,647, and the entire disclosure of this patent is hereby incorporated by reference into this specification. This in vitro culture system of this patent may be used as the precursor for the implantable in vitro capsule described herein. This is only one example of an organ type which can be utilized for the present invention. Additional organ and cellular structures may require very different culture conditions.

Referring again to FIG. 2, and in the preferred embodiment depicted therein, blood is withdrawn via a catheter (not shown) from venous blood supply 12 to blood analyzer 18 via pump 62. After such blood is analyzed, it is returned to blood supply 12 via line 64. In one embodiment, this process is continuous.

The information obtained from the blood analyses is fed via communications line 66 to ASIC controller 22. In one embodiment, in addition to analyzing the hormone levels in the venous blood supply 12, and controlling the amount of analyte released from reservoir 46 (see FIG. 1), the controller 22 preferably controls the type and concentrations of constituents fed into the cell culture system 46 which are necessary for the in vitro production of the desired analytes. These reagents are fed from culture media reservoir 70 to cell culture assembly 46 via line 72 in response to signals from controller 22 which are fed back and forth via line 74.

The reagents which are fed from culture media reservoir 70 are initially collected in culture media collector. The controller 22 furnishes information to collector 76 via line 78 as to the type and concentration of the various analytes which are required for the maintenance of the in vitro cell culture system 46. These analytes are initially fed to collector 76 via line 80 and, thereafter, it is passed via line 82 to filter 84, in which the analytes are sterilized and purified.

As is known to those in the art of cell culturing, the filter removes bacteria, immunogens, and other agents which are not conducive for the desired in vitro cell culture processes.

In one embodiment, the pH of the material in the cell culture reservoir 70 is monitored to insure that it preferably is between 7.1 to 7.4. If the pH measured in reservoir 70 is lower than this range, controller 22 signals culture media collector 76 to extract carbonic anhydrase (carbonic acid minus a hydrogen ion) from venous blood supply 12 to feed it to filter 84 and thence to culture media reservoir 70, where its presence will increase the pH. Conversely, if the pH in reservoir 70 is higher than the desired range, carbonic anhydrase may be withdrawn from the reservoir 70.

In a similar manner, the pH within the cell culture assembly 46, and within each of the compartments 51, 53, and 55 thereof, may also be adjusted by the addition or removal of the carbonic anhydrase, in response to signals from the controller 22 (see line 57). In this embodiment, the carbonic anhydrase may be fed via line 72 to cell culture assembly 46 and/or any component thereof.

As will be apparent, there are several information streams fed into the controller 22, including streams of the information about the pH in both reservoir 70 and cell culture assembly 46. The controller 22 evaluates all of these factors and then determines precisely what mix of reagents is needed to feed via lines 80, 82, and 72 to obtain the desired pH range (and anayltes) in both culture reservoir 70 and cell culture assembly 46. In addition to the carbonic anhydrase, the controller 22 may feed other pH-modifying analytes to adjust the pH.

Referring again to FIG. 2, the analytes required by the body to maintain the desired homoestasis condition(s) are withdrawn, as needed, from cell culture assembly 46 by pump 90 and fed via line 92 to isolator assembly 94.

Isolator assembly 94 is comprised of a multiplicity of isolation filter columns 96, 98, 100 and 102, which, by appropriate purification and elution techniques, isolate one or more purified analytes from each of columns 96, 98, 100, and 102 et seq. The purified analytes are then fed, as needed, via line 104 to reservoir assembly 106, in which one or more of the purified analytes may be separately stored in reservoir chambers 108, 110, 112, 114 et seq. Based upon the directions received from controller 22, these purified analytes maybe fed into venous blood supply 12 via line 116.

In one embodiment, the analyte(s) in each of reservoir chambers 108, 110, 112, and 114 are diluted in a separate dilution chamber (not shown) disposed within each such reservoir. It is preferred that the analyte(s) be diluted with blood plasma, which contains neither red blood cells nor white blood cells.

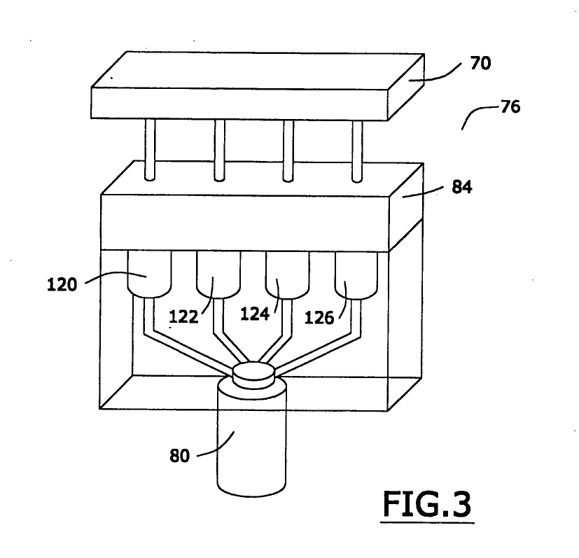

FIG. 3 is schematic view of a preferred embodiment of culture media collector 76. Referring to FIG. 3, it will be seen that collector 76 is comprised of input port 80 which communicates with filter banks 120, 122, 124, and 126. Although only four such filter banks, and associated lines, are illustrated in FIG. 3, it will be apparent that many more (or fewer) filter banks can be used, depending upon the number of analytes involved.

In one embodiment, the filter banks 120 et seq. are immunoisolation chambers or columns. In another embodiment, one or more of the purification techniques disclosed in Terry M. Phillips et al.'s “Affinity and Immunoaffinity” (Eaton Publishing, 2000) may be used.

The purified outputs from banks 120 et seq. are then fed to filter 84 and thence to culture media reservoir 70.

The device 76, in addition to being used as culture media collector 76, may also be used as the isolator bank 94 and/or as a component of the blood analyzer 18.

The processes and devices disclosed in this specification may be used with a multiplicity of different organ systems. Thus, by way of illustration, it may be used as an implantable dialysis device in the manner discussed in U.S. Pat. No. 5,902,336 to Mishkin (Implantable Device and Method for Removing Fluids from the Blood of a Patient Method for Implanting such a Device and Method for Treating a Patient Experiencing Renal Failure). Thus, e.g., it may be used as an implantable liver, an implantable bladder (see U.S. Pat. No. 4,961,747 to Wascher (Implantable Artificial Bladder System)), an implantable thymus, an implantable adrenal medulla, and like. By way of further illustration, the devices and processes of this application may be used for the enhancement of T-cell production in immune disorders, for the enhancement of Hepatic function for various liver, disorders, for the enhancement of renal function for various kidney disorders, for the enhancement of digestive function in any number of digestive system disorders, for the enhancement of reproductive function in any number of reproductive system disorders, for the enhancement of cardiac function in any number of cardiac disorders, etc.

In one embodiment, the artificial organ of this invention is hermetically sealed entirely to prevent corrosion. It preferred to seal the artificial organ with a biocompatible coating. In an additional embodiment, the enclosed invention may also be used for the early stage detection of tumorigenic and/or metastatic conditions.

In yet another embodiment of this invention the reduction in specific enzymes required for an efficient and homeostatic physiological condition is detected. Such enzymes are responsible for and/or a product of any and all combinations of efficient physiological function.

It is to be understood that the aforementioned description is illustrative only and that changes can be made in the apparatus, in the ingredients and their proportions, and in the sequence of combinations and process steps, as well as in other aspects of the invention discussed herein, without departing from the scope of the invention as defined in the followings claims.

1. An artificial organ comprising:
a. a conduit adapted to be connected to a blood pool, said blood pool comprising a first analyte and a second analyte;
b. a cell culture media comprised of a first cell culture and a second cell culture, said first cell culture being capable of producing said first analyte and said second cell culture being capable of producing said said second analyte; and
c. a controller comprised of means for measuring a concentration of said first analyte in said blood pool, means for measuring a concentration of said second analyte in said blood pool, means for delivering a specified amount of said first analyte from said cell culture media to said blood pool, and means for delivering a specified amount of said second analyte from said cell culture media to said blood pool.

2. The artificial organ as recited in claim 1, wherein said first analyte and said second analyte are selected from the group consisting of insulin, glucagons, somatostatin, and combinations thereof.

3. The artificial organ as recited in claim 1, wherein said blood pool is further comprised of a third analyte, said cell culture media is further comprised of a third cell culture media which produces said third analyte, and said controller is further comprised of means for measuring a concentration of said third analyte in said blood pool, and means for delivering a specified amount of said third analyte from said cull culture media to said blood pool.

4. The artificial organ as recited in claim 3, wherein said first analyte, said second analyte, and said third analyte are selected from the group consisting of insulin, glucagons, somatostatin, and combinations thereof.

5. The artificial organ as recited in claim 4, wherein said controller is an Application Specific Integrated Circuit controller.

6. The artificial organ as recited in claim 4, wherein said cell culture media has a pH between about 7.1 and about 7.4.

7. The artificial organ as recited in claim 4, wherein said means for delivering a specified amount of said first analyte from said cell culture media to said blood pool further comprises a filter for purifying said first analyte.

8. The artificial organ as recited in claim 4, wherein said artificial organ is implantable within a living organism.

9. The artificial organ as recited in claim 8, wherein said artificial organ is hermetically sealed.

10. The artificial organ as recited in claim 1, further comprising a reservoir containing at least a portion of said blood pool.

11. A process for adjusting the concentration of an analyte in a living organism comprising the step of disposing the artificial organ as recited in claim 8 within a living organism, wherein said blood pool is comprised of blood from said living organism.

12. A process for adjusting the concentration of an analyte in a living organism comprising the step of connecting the artificial organ as recited in claim 4 to a blood vessel of a living organism, wherein said blood pool is comprised of blood from said living organism and said artificial organ is disposed outside of said living organism.

13. The process as recited in claim 12, wherein said blood vessel is selected from the group consisting of an artery, a vein, and combinations thereof.

14. An artificial organ comprised of:
a. a conduit adapted to be connected to a blood pool, said blood pool comprising a first analyte and a second analyte selected from the group consisting of insulin, glucagon, somatostatin, and combinations thereof;
b. a cell culture media comprised of a first cell culture which produces said first analyte; and
c. a controller comprised of means for measuring a concentration of said first analyte in said blood pool, thus producing a first measured concentration, and means for delivering a specified amount of said first analyte from said cell culture media to said blood pool.

15. The artificial organ as recited in claim 14, wherein said controller determines said specified amount of said first analyte based on said first measured concentration.

16. The artificial organ as recited in claim 15, wherein said blood pool is further comprised of a second analyte, said cell culture media is further comprised of a second cell culture media which produces said second analyte, and said controller is further comprised of means for measuring a concentration of said second analyte in said blood pool, thus producing a second measured concentration, and means for delivering a specified amount of said second analyte from said cull culture media to said blood pool.

17. The artificial organ as recited in claim 16, wherein said controller determines said specified amount of said second analyte based on said second measured concentration.

18. The artificial organ as recited in claim 17, wherein said blood pool is further comprised of a third analyte, said cell culture media is further comprised of a third cell culture media which produces said third analyte, and said controller is further comprised of means for measuring a concentration of said third analyte in said blood pool, thus producing a third measured concentration, and means for delivering a specified amount of said third analyte from said cull culture media to said blood pool.

19. The artificial organ as recited in claim 18, wherein said controller determines said specified amount of said third analyte based on said third measured concentration.

20. The artificial organ as recited in claim 15 further comprising a reservoir containing at least a portion of said blood pool.
